(19) United States
(12) Patent Application Publication
(10) Pub. No.: US 2010/0075415 A1
(43) Pub. Date: Mar. 25, 2010

(54) METHOD FOR REDUCING PROTEASE ACTIVITY IN PLANT HYDROLYSATE

(75) Inventor: Kimberly M. May, Scotch Plains, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: SCHERING CORPORATION, Kenilworth, NJ (US)

(21) Appl. No.: 12/514,608

(22) PCT Filed: Nov. 13, 2007
(86) PCT No.: PCT/US2007/023773

§ 371 Date: Sep. 29, 2009

Related U.S. Application Data
(60) Provisional application No. 60/858,522, filed on Nov. 13, 2006.

(51) Int. Cl. C12N 5/071

(52) U.S. Cl. 435/358

(57) ABSTRACT

The present invention provides methods for reducing protease activity in plant hydrolysates that are utilized in cell culture media. The method involves a combination of heating and ultrafiltration to remove proteases present in the hydrolysate prior to the addition of the hydrolysate to the cell culture medium.

### FIELD OF THE INVENTION

The present invention provides methods for reducing protease activity in plant hydrolysates that are utilized in cell culture media.

### BACKGROUND OF THE INVENTION

Mammalian cell culture is used to produce recombinant proteins, for example, recombinant therapeutic proteins. Protein hydrolysates are sometimes added to cell culture media in order to increase the production of intact recombinant protein. A protein hydrolysate is produced by treating a protein source, e.g., soy, wheat or rice, with an exogenous protease. A protease is an enzyme that digests, i.e., cleaves, proteins in the protein source, to form peptides, which serve as a source of amino acid building nutrients for the host cells in culture to produce the recombinant protein.

Plants contain natural proteases. However, to create a hydrolysate, it is necessary to treat the plant material with an exogenous protease, such as papain or a papain derivative. The protease is added to the plant material, and is allowed to digest the protein in the plant material for a specific amount of time, thereby forming a protein hydrolysate. The protein hydrolysate, which contains the exogenous protease and endogenous plant proteases, is typically then heat-treated via a pasteurization step. The protein hydrolysate is then added to cell culture medium.

One drawback to using plant hydrolysates in cell culture media is that, if the endogenous plant protease and/or the exogenous protease are not removed from the plant hydrolysate prior to the addition of the hydrolysate to the cell culture medium, the recombinant protein may be cleaved, resulting in a decrease in intact recombinant protein yield.

For high-throughput production of plant hydrolysate, one approach to inactivating the protease is to pass the hydrolysate through a heat exchanger, which is a heated surface, e.g., a pipe or tube, through which the hydrolysate is pumped. Contact of the liquid hydrolysate along the heat exchanger surface is intended to heat the hydrolysate to a temperature high enough, and for a length of time long enough, to heat-inactivate the protease and prevent microbial contamination. However, the contact time is typically too short, such that not all of the protease is inactivated.

Even if the hydrolysate is subsequently ultrafiltered using filtration cutoff membranes ranging in size from 10 kDa to 50 kDa, some enzymes are not removed. In addition, assays commonly used to detect protease activity do not reliably detect the presence of proteases.

Thus, there is a need in the art to obtain a reliable way to remove proteases from plant hydrolysates, in order to maximize the production of intact recombinant protein by cells in culture.

### SUMMARY OF THE INVENTION

The present invention provides a method for reducing protease activity in a cell culture medium containing a protein hydrolysate. In a preferred embodiment, the method comprises the steps of: (a) heating the hydrolysate at more than one temperature for more than about 5 minutes; and (b) ultrafiltering the hydrolysate through one or more membranes that filter out particles having a molecular weight greater than 10 kDa; wherein the heating and ultrafiltering are performed prior to the addition of the hydrolysate to a cell culture medium. In a preferred embodiment, the protein hydrolysate is a soy or wheat hydrolysate. In one embodiment, the ultrafiltering step uses at least one membrane that filters out particles having molecular weight greater than 5 KDa. In another embodiment, the ultrafiltering step uses at least one membrane that filters out particles having molecular weight greater than 10 KDa.

In one embodiment, the invention provides a method for reducing protease activity in a cell culture medium containing a soy hydrolysate, comprising the step of heating the soy hydrolysate at more than one temperature for more than about 5 minutes, wherein the heating is performed prior to the addition of the soy hydrolysate to a cell culture medium.

In another embodiment, the method comprises two or more steps selected from the group consisting of:
(a) heating the soy hydrolysate at about 90° C. for about 10 to about 45 minutes;
(b) heating the soy hydrolysate at about 100° C. for about 3 to about 10 minutes;
(c) heating the soy hydrolysate at about 105° C. for zero to about 5 minutes;
(d) heating the soy hydrolysate at about 110° C. for zero to about 2 minutes;
(d) heating the soy hydrolysate at about 129° C. for about 15 seconds;
(e) heating the soy hydrolysate at about 107° C. for about 15 seconds; and
(f) heating the soy hydrolysate at about 149° C. for about 20 seconds to about 2 minutes. In one embodiment, step (f) comprises heating the soy hydrolysate at about 149° C. for about 20 seconds. In another embodiment, step (f) comprises heating the soy hydrolysate at about 149° C. for about 40 seconds. In another embodiment, step (f) comprises heating the soy hydrolysate at about 149° C. for about 60 seconds. In another embodiment, step (f) comprises heating the soy hydrolysate at about 149° C. for about 90 seconds.

In another embodiment, the method comprises two or more steps selected from the group consisting of:
(a) heating the soy hydrolysate at about 90° C. for about 10 minutes;
(b) heating the soy hydrolysate at about 100° C. for about 3 minutes;
(c) heating the soy hydrolysate at about 110° C. for about 2 minutes;
(d) heating the soy hydrolysate at about 129° C. for about 15 seconds;
(e) heating the soy hydrolysate at about 107° C. for about 15 seconds; and
(f) heating the soy hydrolysate at about 149° C. for about 20 seconds.

In another embodiment, the method comprises two or more steps selected from the group consisting of:
(a) heating the soy hydrolysate at about 90° C. for about 15 minutes;
(b) heating the soy hydrolysate at about 100° C. for about 10 minutes;
(c) heating the soy hydrolysate at about 105° C. for about 5 minutes;
(d) heating the soy hydrolysate at about 129° C. for about 15 seconds;
(e) heating the soy hydrolysate at about 107° C. for about 15 seconds; and
(f) heating the soy hydrolysate at about 149° C. for about 20 seconds.

In another embodiment, the method comprises two or more steps selected from the group consisting of:
(a) heating the soy hydrolysate at about 90° C. for about 30 minutes;
(b) heating the soy hydrolysate at about 100° C. for about 10 minutes;
(c) heating the soy hydrolysate at about 105° C. for about 5 minutes;
(d) heating the soy hydrolysate at about 129° C. for about 15 seconds;
(e) heating the soy hydrolysate at about 107° C. for about 15 seconds; and
(f) heating the soy hydrolysate at about 149° C. for about 20 seconds.

In another embodiment, the method comprises two or more steps selected from the group consisting of:
(a) heating the soy hydrolysate at about 90° C. for about 45 minutes;
(b) heating the soy hydrolysate at about 100° C. for about 10 minutes;
(c) heating the soy hydrolysate at about 105° C. for about 5 minutes;
(d) heating the soy hydrolysate at about 129° C. for about 15 seconds;
(e) heating the soy hydrolysate at about 107° C. for about 15 seconds; and
(f) heating the soy hydrolysate at about 149° C. for about 20 seconds.

In another embodiment, the method comprises the steps of:
(a) heating the soy hydrolysate at about 90° C. for about 10 minutes; followed by
(b) heating the soy hydrolysate at about 100° C. for about 3 minutes; followed by
(c) heating the soy hydrolysate at about 110° C. for about 2 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds.

In another embodiment, the method comprises the steps of:
(a) heating the soy hydrolysate at about 90° C. for about 15 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds.

In another embodiment, the method comprises the steps of:
(a) heating the soy hydrolysate at about 90° C. for about 30 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds.

In another embodiment, the method comprises the steps of:
(a) heating the soy hydrolysate at about 90° C. for about 45 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds.

In a preferred embodiment, the method comprises the step of heating the soy hydrolysate (e.g., as described above), and further comprises the step of ultrafiltering the hydrolysate through a membrane, wherein said membrane filters out particles having a molecular weight greater than 10 kDa; wherein the heating and ultrafiltering are performed prior to the addition of the hydrolysate to a cell culture medium. In another embodiment, the membrane filters out particles having a molecular weight greater than 5 kDa.

In an exemplary embodiment, the method comprises the step of heating the soy hydrolysate (e.g., as described above), and further comprises the step of ultrafiltering the hydrolysate through two or more membranes; wherein the heating and ultrafiltering are performed prior to the addition of the hydrolysate to a cell culture medium. In one embodiment, at least one of the two or more membranes filters out particles having a molecular weight greater than 5 kDa. In another embodiment, one of the two or more membranes filters out particles having a molecular weight greater than 10 kDa, and another of the two or more membranes filters out particles having a molecular weight greater than 5 kDa.

The present invention also provides a method for reducing protease activity in a cell culture medium containing a wheat hydrolysate, In a preferred embodiment, the method comprises the steps of (a) heating the wheat hydrolysate at about 100° C. to about 120° C. for about 1 to about 5 minutes; and (b) ultrafiltering the wheat hydrolysate through one or more low molecular weight membranes, wherein the heating and ultrafiltering are performed prior to the addition of the wheat hydrolysate to a cell culture medium.

In one embodiment, the invention provides a method for reducing protease activity in a cell culture medium containing a wheat hydrolysate, comprising the step of heating the wheat hydrolysate for one or more minutes prior to the addition of the wheat hydrolysate to a cell culture medium. In another embodiment, the method comprises the step of heating the wheat hydrolysate at about 100° C. to about 120° C. for about 1 to 10 minutes. In another embodiment, the method comprises the step of heating the wheat hydrolysate at about 100° C. for about 1 to 10 minutes. In another embodiment, the method comprises the step of heating the wheat hydrolysate at about 120° C. for about 1 to 10 minutes.

In a preferred embodiment, the method comprises the step of heating the wheat hydrolysate (e.g., as described above), and further comprises the step of ultrafiltering the hydrolysate through a membrane, wherein said membrane filters out particles having a molecular weight greater than 10 kDa; wherein the heating and ultrafiltering are performed prior to the addition of the hydrolysate to a cell culture medium. In another embodiment, the membrane filters out particles having a molecular weight greater than 5 kDa.

In an exemplary embodiment, the method comprises the step of heating the wheat hydrolysate (e.g., as described above), and further comprises the step of ultrafiltering the hydrolysate through two or more membranes; wherein the heating and ultrafiltering are performed prior to the addition of the hydrolysate to a cell culture medium. In one embodiment, at least one of the two or more membranes filters out particles having a molecular weight greater than 5 kDa. In another embodiment, one of the two or more membranes filters out particles having a molecular weight greater than 10 kDa, and another of the two or more membranes filters out particles having a molecular weight greater than 5 kDa.

The invention also provides method for reducing protease activity in a cell culture medium containing a hydrolysate, comprising the step of ultrafiltering the hydrolysate through a low molecular weight membrane, wherein said membrane filters out particles having a molecular weight greater than 5 kDa; wherein the ultrafiltering is performed prior to the addition of the hydrolysate to a cell culture medium.

The invention also provides a method for reducing protease activity in a cell culture medium containing a hydrolysate, comprising the step of ultrafiltering the hydrolysate through two or more low molecular weight membranes; wherein the ultrafiltering is performed prior to the addition of the hydrolysate to a cell culture medium. In one embodiment, the method comprises the step of ultrafiltering the hydrolysate through at least one membrane that filters out particles having a molecular weight greater than 5 kDa. In another embodiment, the method comprises the step of ultrafiltering the hydrolysate through two membranes, wherein the first membrane filters out particles having a molecular weight greater than 10 kDa, and wherein the second membrane filters out particles having a molecular weight greater than 5 kDa.

In one embodiment, the invention provides a method for reducing protease activity in a cell culture medium containing a soy hydrolysate, comprising the steps of:
(a) heating the soy hydrolysate at about 90° C. for about 10 minutes; followed by
(b) heating the soy hydrolysate at about 100° C. for about 3 minutes; followed by
(c) heating the soy hydrolysate at about 110° C. for about 2 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a membrane that filters out particles having a molecular weight greater than 10 kDa;
wherein the heating and ultrafiltering are performed prior to the addition of the soy hydrolysate to a cell culture medium.

In one embodiment, the invention provides a method for reducing protease activity in a cell culture medium containing a soy hydrolysate, comprising the steps of:
(a) heating the soy hydrolysate at about 90° C. for about 10 minutes; followed by
(b) heating the soy hydrolysate at about 100° C. for about 3 minutes; followed by
(c) heating the soy hydrolysate at about 110° C. for about 2 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a membrane that filters out particles having a molecular weight greater than 5 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 10 minutes; followed by
(b) heating the soy hydrolysate at about 100° C. for about 3 minutes; followed by
(c) heating the soy hydrolysate at about 110° C. for about 2 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a first membrane that filters out particles having a molecular weight greater than 10 kDa; followed by
(h) ultrafiltering the soy hydrolysate through a second membrane that filters out particles having a molecular weight greater than 5 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 15 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a membrane that filters out particles having a molecular weight greater than 10 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 15 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a membrane that filters out particles having a molecular weight greater than 5 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 15 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a first membrane that filters out particles having a molecular weight greater than 10 kDa; followed by
(h) ultrafiltering the soy hydrolysate through a second membrane that filters out particles having a molecular weight greater than 5 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 30 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a membrane that filters out particles having a molecular weight greater than 10 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 30 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a membrane that filters out particles having a molecular weight greater than 5 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 30 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a first membrane that filters out particles having a molecular weight greater than 10 kDa; followed by
(h) ultrafiltering the soy hydrolysate through a second membrane that filters out particles having a molecular weight greater than 5 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 45 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a membrane that filters out particles having a molecular weight greater than 10 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 45 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a membrane that filters out particles having a molecular weight greater than 5 kDa;
wherein the heating and ultrafiltering are performed prior to the addition of the soy hydrolysate to a cell culture medium.

The invention also provides a method for reducing protease activity in a cell culture medium containing a soy hydrolysate, comprising the steps of:
(a) heating the soy hydrolysate at about 90° C. for about 45 minutes; followed by
(b) heating the soy hydrolysate at about 100° C. for about 10 minutes; followed by
(c) heating the soy hydrolysate at about 105° C. for about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds; followed by
(g) ultrafiltering the soy hydrolysate through a first membrane that filters out particles having a molecular weight greater than 10 kDa; followed by
(h) ultrafiltering the soy hydrolysate through a second membrane that filters out particles having a molecular weight greater than 5 kDa;
wherein the heating and ultrafiltering are performed prior to the addition of the soy hydrolysate to a cell culture medium.

The invention also provides a method for reducing protease activity in a cell culture medium containing a wheat hydrolysate, comprising the steps of:
(a) heating the wheat hydrolysate at about 100° C. for about 1 to 10 minutes; followed by (b) ultrafiltering the wheat hydrolysate through a membrane that filters out particles having a molecular weight greater than 10 kDa;
wherein the heating and ultrafiltering are performed prior to the addition of the wheat hydrolysate to a cell culture medium.

The invention also provides a method for reducing protease activity in a cell culture medium containing a wheat hydrolysate, comprising the steps of:
(a) heating the wheat hydrolysate at about 100° C. for about 1 to 10 minutes; followed by (b) ultrafiltering the wheat hydrolysate through a membrane that filters out particles having a molecular weight greater than 5 kDa;
wherein the heating and ultrafiltering are performed prior to the addition of the wheat hydrolysate to a cell culture medium.

The invention also provides a method for reducing protease activity in a cell culture medium containing a wheat hydrolysate, comprising the steps of:
(a) heating the wheat hydrolysate at about 100° C. for about 1 to 10 minutes; followed by (b) ultrafiltering the wheat hydrolysate through a first membrane that filters out particles having a molecular weight greater than 10 kDa; followed by
(c) ultrafiltering the wheat hydrolysate through a second membrane that filters out particles having a molecular weight greater than 5 kDa;
wherein the heating and ultrafiltering are performed prior to the addition of the wheat hydrolysate to a cell culture medium.

The invention also provides a method for reducing protease activity in a cell culture medium containing a wheat hydrolysate, comprising the steps of:
(a) heating the wheat hydrolysate at about 120° C. for about 1 to 10 minutes; followed by (b) ultrafiltering the wheat hydrolysate through a membrane that filters out particles having a molecular weight greater than 10 kDa;
wherein the heating and ultrafiltering are performed prior to the addition of the wheat hydrolysate to a cell culture medium.

The invention also provides a method for reducing protease activity in a cell culture medium containing a wheat hydrolysate, comprising the steps of:
(a) heating the wheat hydrolysate at about 120° C. for about 1 to 10 minutes; followed by (b) ultrafiltering the wheat hydrolysate through a membrane that filters out particles having a molecular weight greater than 5 kDa;
wherein the heating and ultrafiltering are performed prior to the addition of the wheat hydrolysate to a cell culture medium.

The invention also provides a method for reducing protease activity in a cell culture medium containing a wheat hydrolysate, comprising the steps of:
(a) heating the wheat hydrolysate at about 120° C. for about 1 to 10 minutes; followed by (b) ultrafiltering the wheat hydrolysate through a first membrane that filters out particles having a molecular weight greater than 10 kDa; followed by
(c) ultrafiltering the wheat hydrolysate through a second membrane that filters out particles having a molecular weight greater than 5 kDa;
wherein the heating and ultrafiltering are performed prior to the addition of the wheat hydrolysate to a cell culture medium.

### BRIEF DESCRIPTION OF THE DRAWINGS

FIG. 1 shows a reducing SDS-PAGE gel detecting proteolysis of monoclonal antibody substrate 1 (MAB1) after incubation with retentate (Ret) and permeate (Perm) fractions of hydrolysate samples, media, and media supplements that have been ultrafiltered through a 5 kDa membrane. As shown, enzymatic activity is mostly concentrated in the retentate (the fraction rejected by the membrane), and is reduced or eliminated in the permeate (the fraction that passes through the membrane).

FIG. 2 shows a non-reducing SDS-PAGE gel detecting proteolysis of monoclonal antibody substrate 1 (MAB1) after incubation with retentate (Ret) and permeate (Perm) fractions of hydrolysates, media, and media supplements that have been ultrafiltered through a 5 kDa membrane. As shown, enzymatic activity is mostly concentrated in the retentate (the fraction rejected by the membrane), and is reduced or eliminated in the permeate (the fraction that passes through the membrane).

FIG. 3 shows a gelatin zymogram that detects the presence of multiple proteases in media and supplement retentate fractions (Ret).

FIG. 4 shows a casein zymogram that detects the presence of multiple proteases in media and supplement retentate fractions (Ret).

FIG. 5 shows a casein zymogram that detects the presence of multiple proteases in the retentate (Ret) of hydrolysate samples from Company 1.

FIG. 6 shows a casein zymogram inhibition study that detects the presence of multiple proteases of the thiol-metalloprotease and metalloprotease families in the retentate (Ret) of hydrolysate samples from Company 3.

FIG. 7 shows an SDS-PAGE inhibition study that detects the presence of a thiol-protease in the retentate (Ret) of lot 1 hydrolysate samples from Company 2.

FIG. 8 shows an SDS-PAGE inhibition study that detects the presence of a thiol-protease and a metalloprotease in the retentate (Ret) of lot 2 hydrolysate samples from Company 2 and a thiol-metalloprotease in the retentate (Ret) of lot 3 hydrolysate samples from Company 2.

FIG. 9 shows the SEC Post-Peak % at 0, 2, 6, 10, and 16 days by SE-HPLC for monoclonal antibody substance 1 (MAB1) added to media containing 15 g/L of the hydrolysate sample. Hydrolysates containing multiple proteases had the largest effect on post-antibody peaks by SE-HPLC.

FIG. 10 shows the % Post SEC Peak at 0, 2, 7, 10, and 16 days by SE-HPLC for monoclonal antibody substance 2 (MAB2) added to media containing 15 g/L of the hydrolysate sample. Hydrolysates containing multiple proteases had the largest effect on post-antibody peaks by SE-HPLC.

FIG. 11 shows the % Post-Antibody Peaks at 0, 2, 6, 10, and 16 days by RP-HPLC for monoclonal antibody substance 1 (MAB1) added to media containing 15 g/L of the hydrolysate sample. Hydrolysates containing multiple proteases had the largest effect on post-antibody peaks by RP-HPLC.

FIG. 12 shows the % Post-Antibody Peaks at 0, 2, 7, 10, and 16 days by RP-HPLC for monoclonal antibody substance 2 (MAB2) added to media containing 15 g/L of the hydrolysate sample. Hydrolysates containing multiple proteases had the largest effect on post-antibody peaks by RP-HPLC.

FIG. 13 shows the fragment % (peaks that elute before the light chain) at 0, 2, 6, 10, and 16 days by RP-HPLC for monoclonal antibody substance 1 (MAB1) added to media containing 15 g/L of the hydrolysate sample. Hydrolysates containing multiple proteases had the largest effect on the fragment eluting out before the light chain.

FIG. 14 shows the fragment % (peaks that elute before the light chain) at 0, 2, 7, 10, and 16 days by RP-HPLC for monoclonal antibody substance 2 (MAB2) added to media containing 15 g/L of the hydrolysate sample. Hydrolysates containing multiple proteases had the largest effect on the fragment eluting out before the light chain.

### DETAILED DESCRIPTION

A “protease” is an enzyme that cleaves a peptide, polypeptide or protein. An “endoprotease” is an enzyme that cleaves a peptide, polypeptide, or protein at an internal site. An “exoprotease” is an enzyme that cleaves a peptide, polypeptide, or protein from one end.

The term “protease activity” refers to proteolysis, i.e., cleavage of a peptide, polypeptide or protein. Proteolysis is readily determined by one of ordinary skill in the art using known methods for analyzing whether the peptide, polypeptide or protein has been cleaved by an endoprotease or by an exoprotease.

The term “plant hydrolysate” refers to a preparation of plant material that has been digested by one or more proteases. In a preferred embodiment, the plant hydrolysate is a soy hydrolysate. In another preferred embodiment, the plant hydrolysate is a rice hydrolysate. In another preferred embodiment, the plant hydrolysate is a wheat hydrolysate. In another preferred embodiment, the plant hydrolysate is a combination of plant hydrolysates from one or more different plants.

The term “ultrafiltration” refers to a separation process by which material is filtered through a medium, such as a membrane, to filter out particles greater than a certain molecular weight. For example, 10 kDa ultrafiltration utilizes a membrane that filters out particles having a molecular weight greater than 10 kilodaltons (“kDa”). Alternatively, 5 kDa ultrafiltration utilizes a membrane that filters out particles having a molecular weight greater than 5 kDa. The term “permeate” refers to the fraction that passes through the membrane. The term “retentate” refers to the fraction that is rejected by the membrane.

As used herein, the term “about” when used in reference to a specified temperature refers to the specified temperature plus or minus 5° C. The term “about” when used in reference to a specified number of seconds refers to the specified number of seconds plus or minus 10 seconds. The term “about” when used in reference to a specified number of minutes refers to the specified number of minutes plus or minus 5 minutes.

A “recombinant” peptide, polypeptide or protein is one that is expressed in a host cell using recombinant expression technology. In a preferred embodiment, the recombinant protein is an antibody or an antigen-binding fragment thereof.

A “host cell” is a cell that harbors a recombinant expression construct that contains nucleic acid that encodes a recombinant peptide, polypeptide or protein. The host cell can be any type of cell that is used to express the recombinant peptide, polypeptide or protein. Preferably, the host cell is a eukaryotic cell. More preferably, the host cell is a mammalian cell. More preferably, the host cell is a Chinese hamster ovary (“CHO”) cell.

The present invention provides methods for reducing protease activity in cell culture media. In one embodiment, the protease activity is reduced by 25%. In another embodiment, the activity is reduced by 50%. In another embodiment, the protease activity is reduced by 75%. In another embodiment, the protease activity is reduced by 85%. In other embodiments, the protease activity is reduced by 90%, 95%, 96%, 97%, 98%, 99% or 100%.

In the method of the present invention, the plant hydrolysate can be heated to the specified temperature using any acceptable technique. For example, the heating can be accomplished using a batch heating approach in a suitable vessel. Alternatively, the heating can be accomplished by autoclaving. In another preferred embodiment, the heating can be accomplished using a heat exchanger system.

Some of the methods of the present invention comprise of a cell culture medium comprising hydrolysates. In one embodiment, the ultrafiltering comprises ultrafiltering one or more times through a 10 kDa membrane. In a preferred embodiment, the ultrafiltering comprises ultrafiltering one or more times through a 5 kDa membrane. In another preferred embodiment, the ultrafiltering comprises ultrafiltering one or more times through a 10 kDa membrane, followed by ultrafiltering one or more times through a 5 kDa membrane.

For soy hydrolysate, a preferred heating and ultrafiltration protocol is heating for about 10 minutes at about 90° C., followed by heating for about 3 minutes at about 100° C., followed by heating for about 2 minutes at about 110° C., followed by heating for about 15 seconds at about 129° C., followed by heating for about 15 seconds at about 107° C., followed by heating for about 20 seconds at about 149° C., followed by ultrafiltration through a 10 kDa or a 5 kDa membrane. In a preferred embodiment, a 5 kDa membrane is used for the ultrafiltration.

Another preferred heating and ultrafiltration protocol for soy hydrolysate is heating for about 10 minutes at about 90° C., followed by heating for about 3 minutes at about 100° C., followed by heating for about 2 minutes at about 110° C., followed by heating for about 15 seconds at about 129° C., followed by heating for about 15 seconds at about 107° C., followed by heating for about 20 seconds at about 149° C., followed by ultrafiltration through a 10 kDa membrane, followed by ultrafiltration through a 5 kDa membrane.

Another preferred heating and ultrafiltration protocol for soy hydrolysate is heating for about 15 minutes at about 90° C., followed by heating for about 10 minutes at about 100° C., followed by heating for about 5 minutes at about 105° C., followed by heating for about 15 seconds at about 129° C., followed by heating for about 15 seconds at about 107° C., followed by heating for about 20 seconds at about 149° C., followed by ultrafiltration through a 10 kDa or a 5 kDa membrane. In a preferred embodiment, a 5 kDa membrane is used for the ultrafiltration.

Another preferred heating and ultrafiltration protocol for soy hydrolysate is heating for about 15 minutes at about 90° C., followed by heating for about 10 minutes at about 100° C., followed by heating for about 5 minutes at about 105° C., followed by heating for about 15 seconds at about 129° C., followed by heating for about 15 seconds at about 107° C., followed by heating for about 20 seconds at about 149° C., followed by ultrafiltration through a 10 kDa membrane, followed by ultrafiltration through a 5 kDa membrane.

Another preferred heating and ultrafiltration protocol for soy hydrolysate is heating for about 30 minutes at about 90° C., followed by heating for about 10 minutes at about 100° C., followed by heating for about 5 minutes at about 105° C., followed by heating for about 15 seconds at about 129° C., followed by heating for about 15 seconds at about 107° C., followed by heating for about 20 seconds at about 149° C., followed by ultrafiltration through a 10 kDa or a 5 kDa membrane. In a preferred embodiment, a 5 kDa membrane is used for the ultrafiltration.

Another preferred heating and ultrafiltration protocol for soy hydrolysate is heating for about 30 minutes at about 90° C., followed by heating for about 10 minutes at about 100° C., followed by heating for about 5 minutes at about 105° C., followed by heating for about 15 seconds at about 129° C., followed by heating for about 15 seconds at about 107° C., followed by heating for about 20 seconds at about 149° C., followed by ultrafiltration through a 10 kDa membrane, followed by ultrafiltration through a 5 kDa membrane.

Another preferred heating and ultrafiltration protocol for soy hydrolysate is heating for about 45 minutes at about 90° C., followed by heating for about 10 minutes at about 100° C., followed by heating for about 5 minutes at about 105° C., followed by heating for about 15 seconds at about 129° C., followed by heating for about 15 seconds at about 107° C., followed by heating for about 20 seconds at about 149° C., followed by ultrafiltration through a 10 kDa or a 5 kDa membrane. In a preferred embodiment, a 5 kDa membrane is used for the ultrafiltration.

Another preferred heating and ultrafiltration protocol for soy hydrolysate is heating for about 45 minutes at about 90° C., followed by heating for about 10 minutes at about 100° C., followed by heating for about 5 minutes at about 105° C., followed by heating for about 15 seconds at about 129° C., followed by heating for about 15 seconds at about 107° C., followed by heating for about 20 seconds at about 149° C., followed by ultrafiltration through a 10 kDa membrane, followed by ultrafiltration through a 5 kDa membrane.

In another preferred embodiment, the present invention provides a method for reducing protease activity in a cell culture medium containing a soy hydrolysate, the method comprising: (a) heating the soy hydrolysate at about 90° C. for about 10 to about 45 minutes, followed by heating the soy hydrolysate at about 100° C. for about 3 to about 10 minutes, followed by heating the soy hydrolysate at about 105° C. for zero to about 5 minutes, followed by heating the soy hydrolysate at about 110° C. for zero to about 2 minutes, followed by heating the soy hydrolysate at about 129° C. for about 15 seconds, followed by heating the soy hydrolysate at about 107° C. for about 15 seconds, followed by heating the soy hydrolysate at about 149° C. for about 20 seconds; and (b) ultrafiltering the plant hydrolysate through a 10 kDa or 5 kDa membrane, wherein the heating and ultrafiltering are performed prior to the addition of the soy hydrolysate to a cell culture medium.

For wheat hydrolysate, a preferred heating and ultrafiltration protocol is heating for about 1-10 minutes at about 100° C., followed by ultrafiltration through a 10 kDa or a 5 kDa membrane. In a preferred embodiment, a 5 kDa membrane is used for the ultrafiltration.

Another preferred heating and ultrafiltration protocol for wheat hydrolysate is heating for about 1-10 minutes at about 100° C., followed by ultrafiltration through a 10 kDa membrane, followed by ultrafiltration through a 5 kDa membrane.

For wheat hydrolysate, a preferred heating and ultrafiltration protocol is heating for about 1-10 minutes at about 120° C., followed by ultrafiltration through a 10 kDa membrane.

Another preferred heating and ultrafiltration protocol for wheat hydrolysate is heating for about 1-10 minutes at about 120° C., followed by ultrafiltration through a 10 kDa membrane, followed by ultrafiltration through a 5 kDa membrane.

In another preferred embodiment, the present invention also provides a method for reducing protease activity in a cell culture medium containing a wheat hydrolysate, the method comprising: (a) heating the wheat hydrolysate at about 100° C. to about 120° C. for about 1 to 10 minutes; and (b) ultrafiltering the wheat hydrolysate through a 10 kDa or a 5 kDa membrane, wherein the heating and ultrafiltering are performed prior to the addition of the wheat hydrolysate to a cell culture medium.

In a preferred embodiment of the method of the present invention, the recombinant peptide, polypeptide or protein produced by cells cultured in the cell culture medium that contains the heated and ultrafiltered plant hydrolysate exhibits less proteolysis than the recombinant peptide, polypeptide or protein exhibits when produced by cells cultured in cell culture medium that contains plant hydrolysate that was not heated and ultrafiltered.

Proteolytic activity in a hydrolysate sample may be determined by, for example, incubating the sample with a substrate for said protease for 8 or more hours and determining proteolysis of said substrate. In one embodiment, the method of determining proteolytic activity comprises the steps of: (a) combining the sample with a peptide substrate and, optionally, with a reducing agent; (b) incubating the sample for at least 8 hours at room temperature; and (c) determining proteolysis of the substrate. In an embodiment of the invention, proteolysis is determined by SDS-polyacrylamide gel electrophoresis analysis.

In order that this invention may be better understood, the following examples are set forth. These examples are for purposes of illustration only and are not to be construed as limiting the scope of the invention in any manner.

### EXAMPLE I

### Determination of Proteolytic Activity In Hydrolysate Samples

To determine the efficacy of ultrafiltration in removing proteases from hydrolysate samples, hydrolysates from 3 different source companies (Companies 1-3), supplements (Sigma-Aldrich 1615 CHO medium supplement) and media (Sigma Aldrich 65753 imMEDIAte Advantage™ hydrolysate-free CHO medium) were ultrafiltered through a 5 kDa membrane using a Millipore Pelicon XL Biomax 5, 50 cm² with a Labscale TFF System.

The permeate and retentate samples were then tested for proteolytic activity. Each sample was combined in equal parts with the proteolytic substrate (an antibody) to yield a final concentration of 0.5 mg/ml antibody. These samples were then mixed with equal parts of reducing or non-reducing buffer and loaded onto reducing or non-reducing SDS-PAGE gels, respectively, for a final load of 10-12 micrograms of antibody.

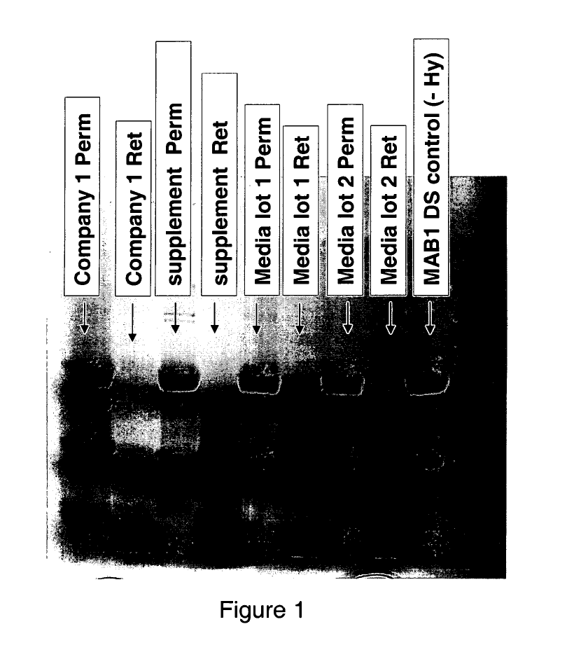

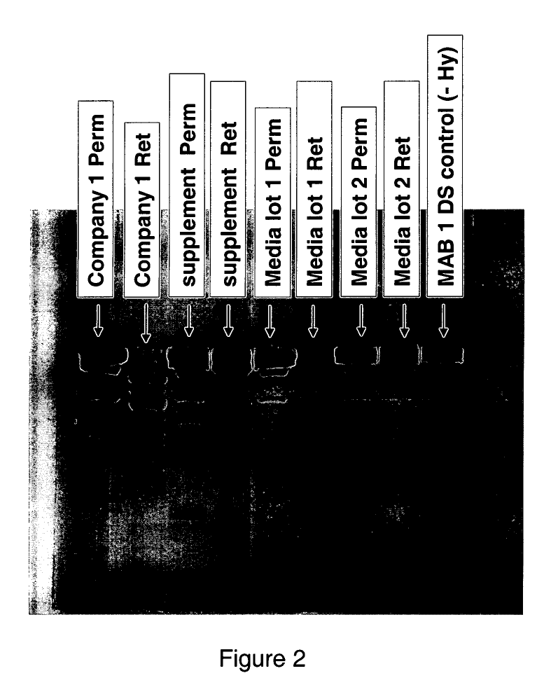

While protease was detected in some permeate samples post-filtration, enzymatic activity was mostly concentrated in the retentate (see, e.g., FIGS. 1 and 2). The data indicates that the 5 kDa ultrafiltration step greatly reduces the protease activity in the permeate. Extensive lot-to-lot variability was observed for all hydrolysates (not shown).

No proteolytic activity was detected in medium without hydrolysates (not shown), thereby indicating that the hydrolysates are the source of proteolytic contamination.

### EXAMPLE II

### Identification of Proteases In Hydrolyasate Samples

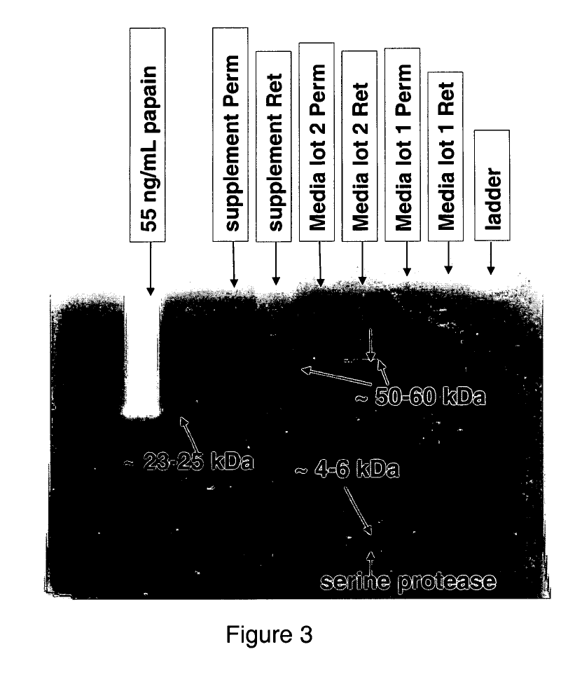

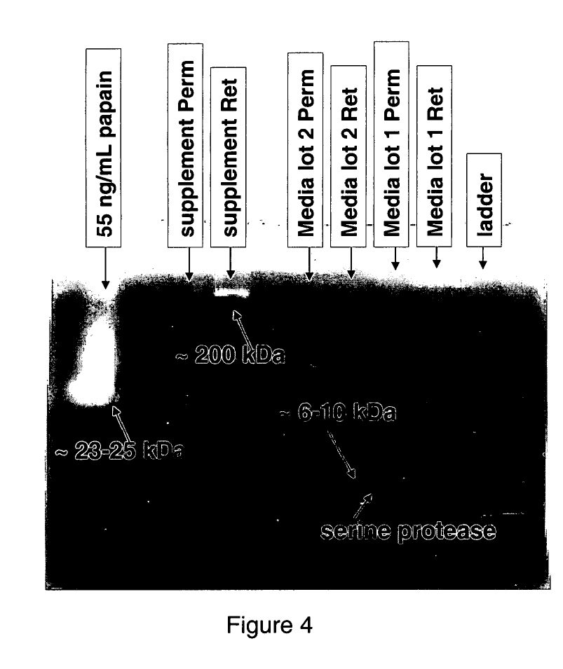

Permeate and retentate samples were also loaded onto gelatin and casein zymogram gels at the maximum volume of 25 microliters and run at a constant voltage of 125 mV for 90 minutes with the XCell Surelock™ Mini-Cell system. The zymograms confirmed the presence of multiple proteases ranging from approximately 4-7 kDa to 200 kDa in the retentate (see, e.g., FIGS. 3 and 4).

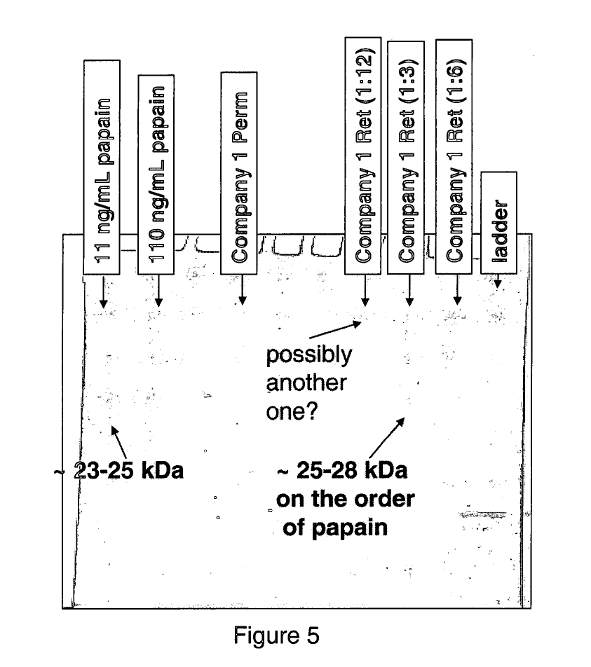

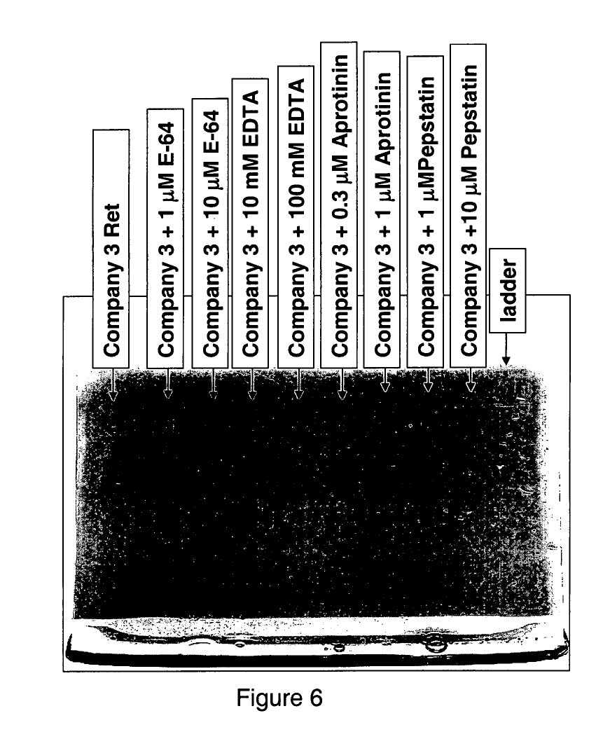

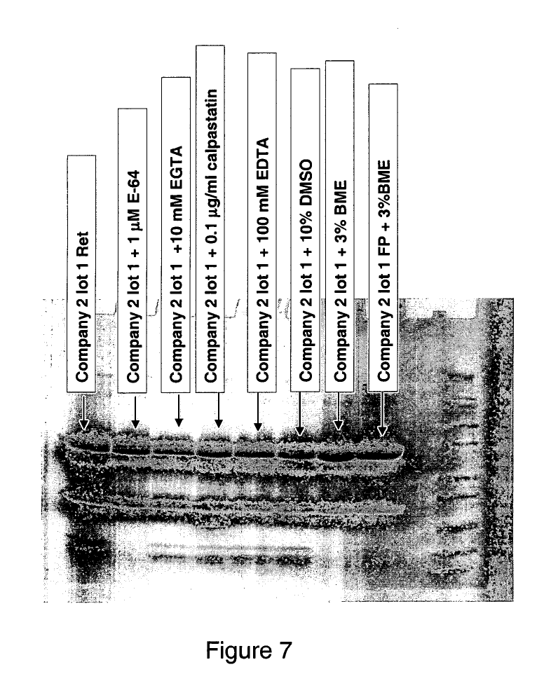

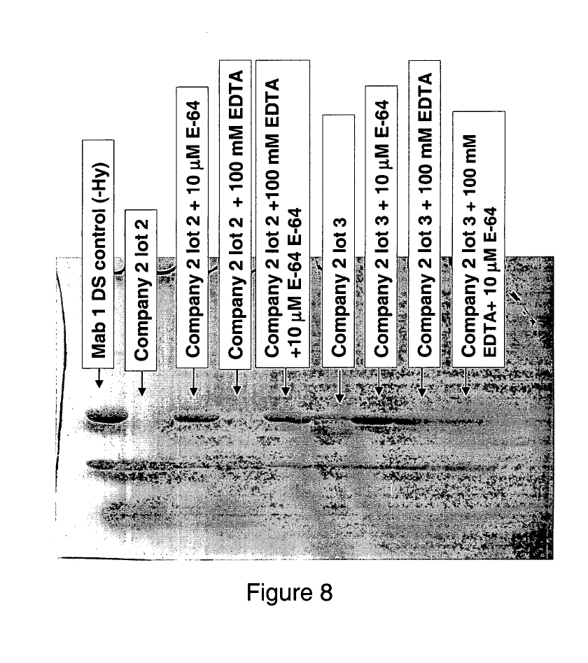

SDS-PAGE and zymogram inhibition studies identified the classes of proteases present within each sample. The studies identified metalloproteases, thiol-proteases, thiol-metalloproteases, and serine proteases ranging from approximately 6 kDa to 200 kDa in molecular weight. The level and composition of proteases in each sample depended on both the source and the lot number. The SDS-PAGE inhibition studies demonstrated that the Company 2 lot 1 hydrolysate sample contains a thiol-protease (see, e.g., FIG. 7); the Company 2 lot 2 hydrolysate sample contains a thiol-protease and a metalloprotease (see, e.g., FIG. 8); the Company 2 lot 3 hydrolysate sample contains a thiol-metalloprotease (see, e.g., FIG. 8); the Company 3-H2 hydrolysate sample contains at least a thiol-protease (not shown); and the media supplement contains an active thiol-protease (not shown). The zymogram inhibition studies demonstrate that the Company 1 hydrolysate sample contains a thiol-protease and likely a metalloprotease as well (see, e.g., FIG. 5); the Company 3-H1 hydrolysate sample contains at least 5 proteases of the thiol-metalloprotease and metalloprotease families (see, e.g., FIG. 6); and the media supplement contains two proteases (not shown).

Proteolysis in samples coordinates with insufficient inactivation and removal of proteases during the manufacturing process. The data demonstrates the extensive variability in protease type and number between hydrolysate samples from different sources and even between hydrolysate lots from the same source.

### EXAMPLE III

### Characterization of Product Quality

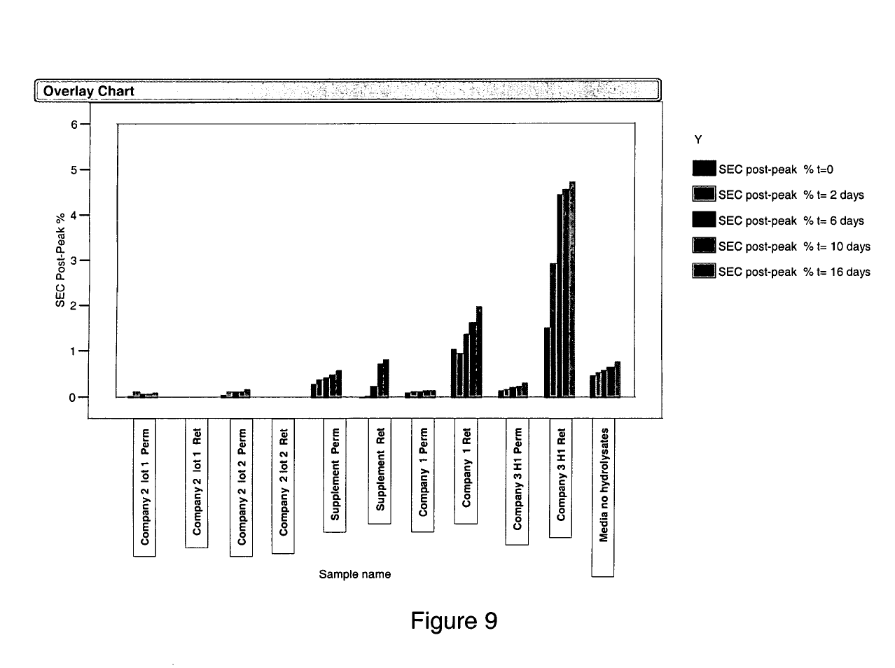

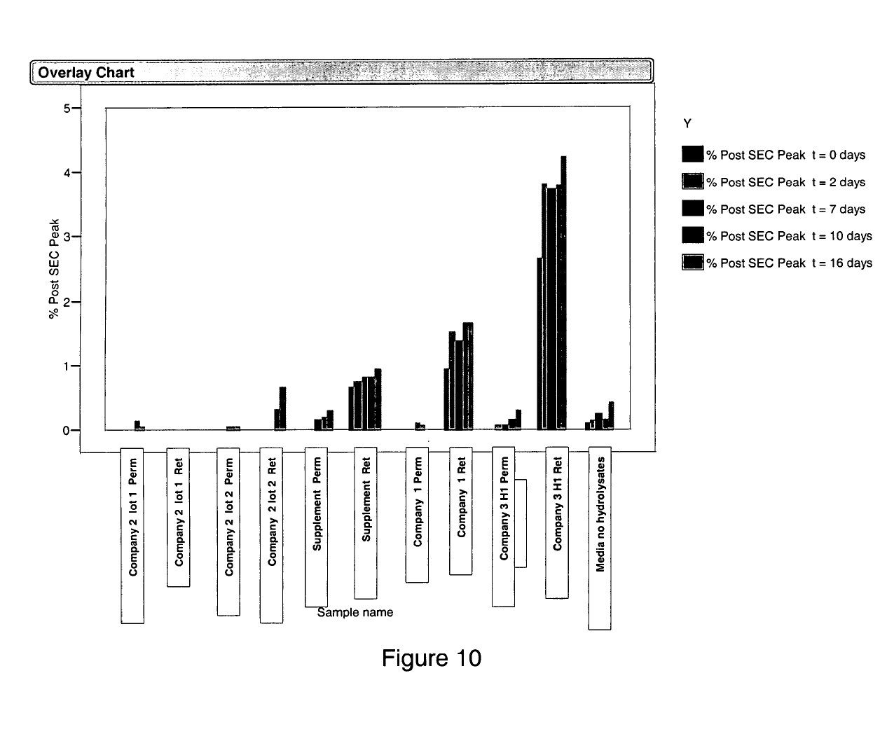

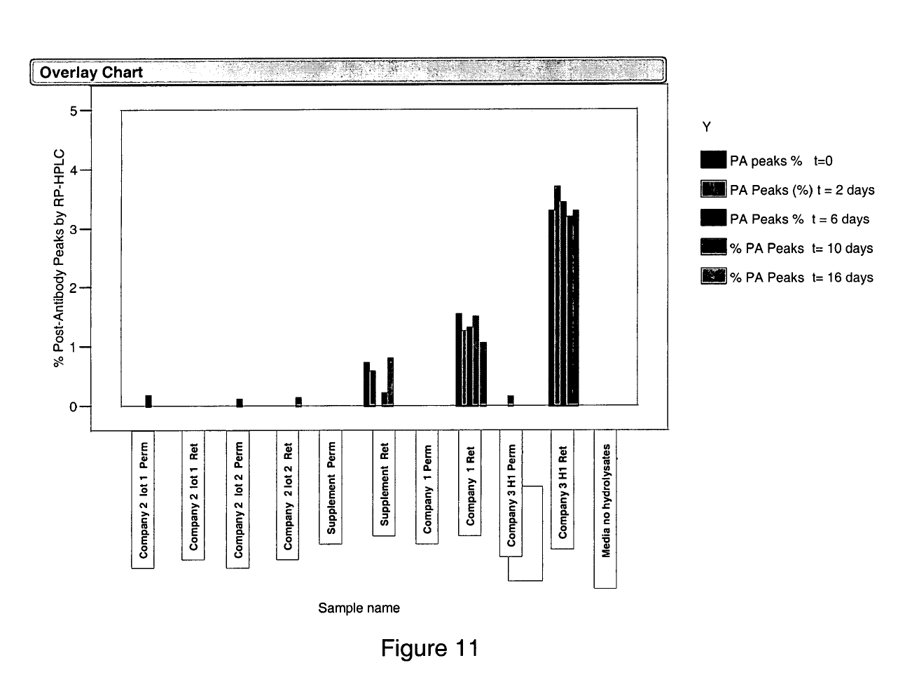

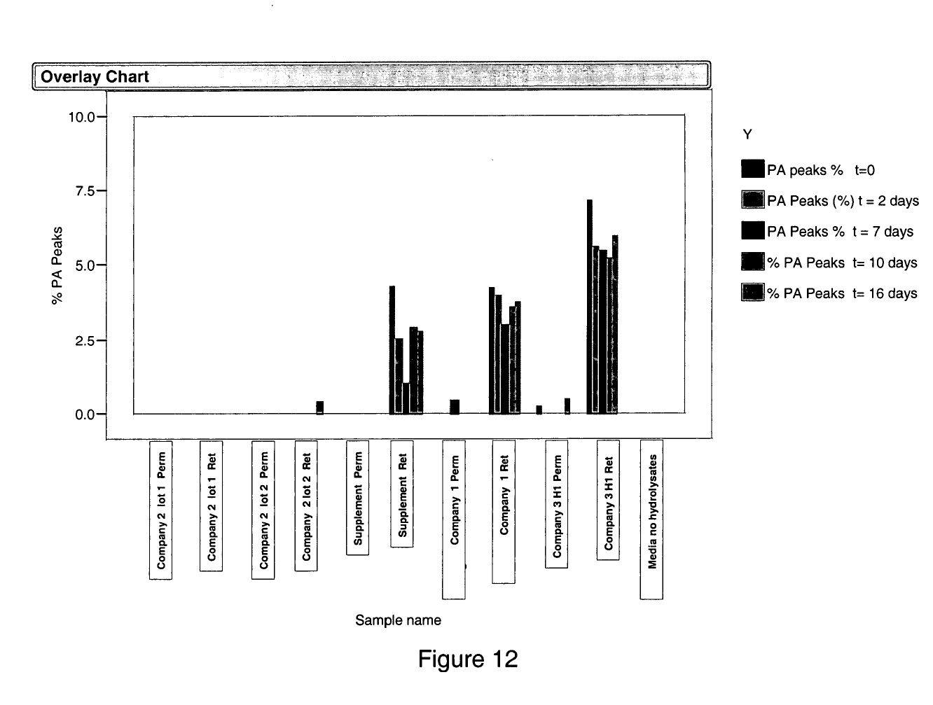

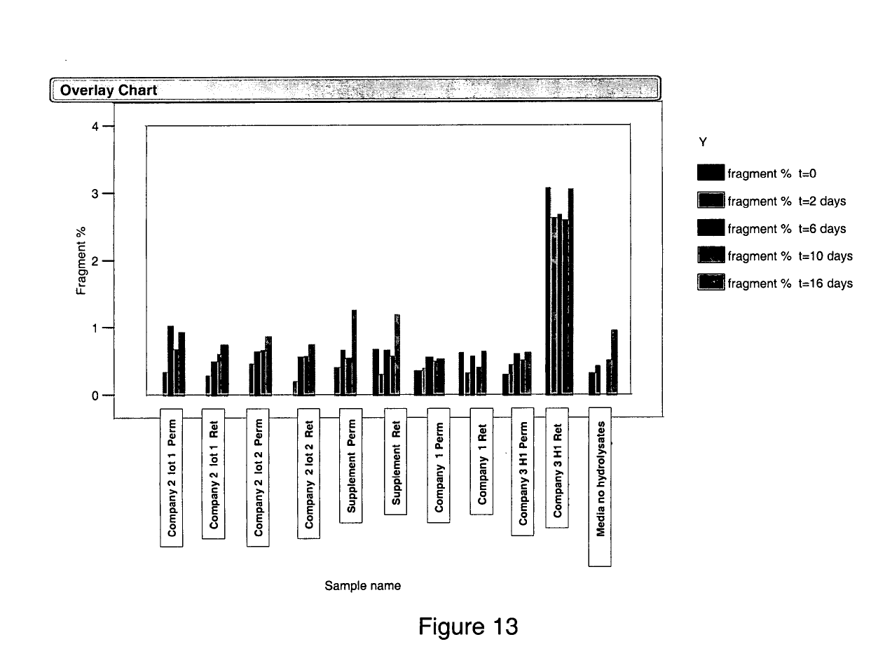

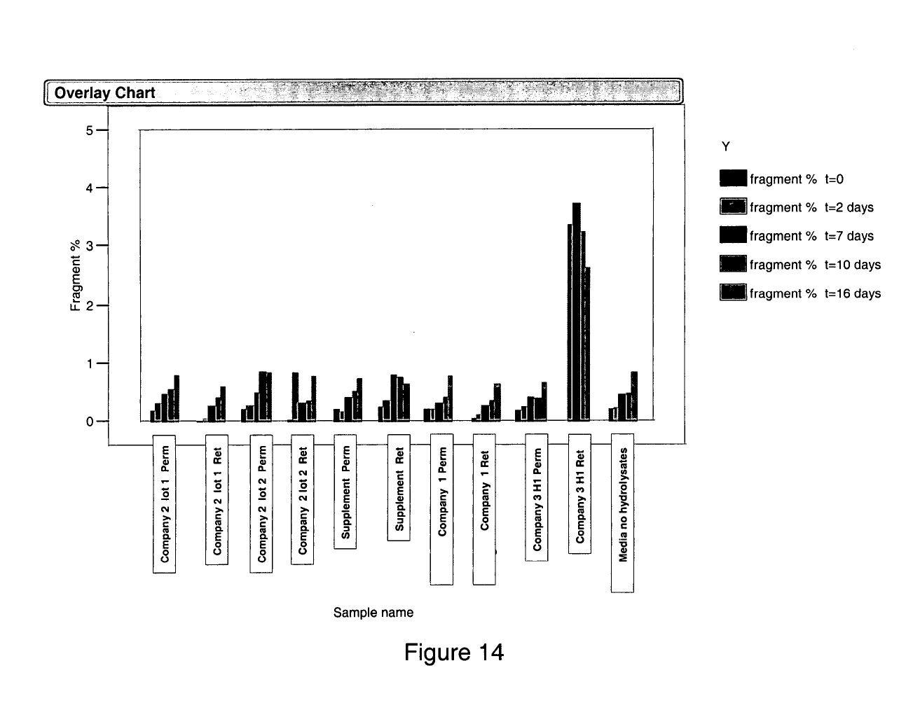

To characterize the product quality of antibodies exposed to the hydrolysate samples, ultrafiltered hydrolysates were added to hydrolysate-free media. Purified antibodies were then added to the solution for a final concentration of 1 mg/mL antibody. Samples were incubated at room temperature to simulate typical native fermentation conditions and monitored periodically over 16 days via size-exchange HPLC (SE-HPLC) and reverse-phase HPLC (RP-HPLC). Hydrolysates containing multiple proteases had the largest effect on post-antibody peaks by SE-HPLC (see, e.g., FIGS. 9 and 10) and by RP-HPLC (see, e.g., FIGS. 11 and 12) and on the level of fragment eluting out before the light chain by RP-HPLC (see, e.g., FIGS. 13 and 14). The Company 3-H1 hydrolysate retentate fraction produced the most post-antibody peaks for both monoclonal antibody substances, followed by the Company 1 hydrolysate retentate fraction and the media supplement retentate fraction. The degradation effects were also antibody-dependent. By RP-HPLC, MAB2 appeared to undergo more degradation than MAB1.

The size of the post-antibody peaks and fragment peaks correlates with a loss of product quality. As shown, the MAB1 or MAB2 incubated with hydrolysate permeate samples was superior in quality to the MAB1 or MAB2 incubated with hydrolysate retentate samples. The data indicates that the 5 kDa ultrafiltration step reduces protease activity in the hydrolysate permeate.

All publications and patent applications cited in this specification are incorporated herein by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for reducing protease activity in a cell culture medium containing a soy hydrolysate, comprising the step of heating the soy hydrosylate at more than one temperature for more than about 5 minutes, wherein the heating is performed prior to the addition of the soy hydrolysate to a cell culture medium.

2. The method according to claim 1, comprising two or more steps selected from the group consisting of:
(a) heating the soy hydrolysate at about 90° C. for about 10 to about 45 minutes;
(b) heating the soy hydrolysate at about 100° C. for about 3 to about 10 minutes;
(c) heating the soy hydrolysate at about 105° C. for zero to about 5 minutes;
(d) heating the soy hydrolysate at about 110° C. for zero to about 2 minutes;
(d) heating the soy hydrolysate at about 129° C. for about 15 seconds;
(e) heating the soy hydrolysate at about 107° C. for about 15 seconds; and
(f) heating the soy hydrolysate at about 149° C. for about 20 seconds.

3. The method according to claim 2, comprising the steps of:
(a) heating the soy hydrolysate at about 90° C. for about 10 to about 45 minutes; followed by
(b) heating the soy hydrolysate at about 100° C. for about 3 to about 10 minutes; followed by
(c) heating the soy hydrolysate at about 105° C. to 110° C. for zero to about 5 minutes; followed by
(d) heating the soy hydrolysate at about 129° C. for about 15 seconds; followed by
(e) heating the soy hydrolysate at about 107° C. for about 15 seconds; followed by
(f) heating the soy hydrolysate at about 149° C. for about 20 seconds.

4. A method for reducing protease activity in a cell culture medium containing a wheat hydrolysate, comprising the step of heating the wheat hydrolysate for one or more minutes prior to the addition of the wheat hydrolysate to a cell culture medium.

5. The method according to claim 4, comprising the step of heating the wheat hydrolysate at about 100° C. to about 120° C. for about 1 to 10 minutes.

6. The method according to claim 5, comprising the step of heating the wheat hydrolysate at about 100° C. for about 1 to 10 minutes.

7. The method according to claim 5, comprising the step of heating the wheat hydrolysate at about 120° C. for about 1 to 10 minutes.

8. A method for reducing protease activity in a cell culture medium containing a hydrolysate, comprising the step of ultrafiltering the hydrolysate through a low molecular weight membrane, wherein said membrane filters out particles having a molecular weight greater than 5 kDa; wherein the ultrafiltering is performed prior to the addition of the hydrolysate to a cell culture medium.

9. A method for reducing protease activity in a cell culture medium containing a hydrolysate, comprising the step of ultrafiltering the hydrolysate through two or more low molecular weight membranes, wherein at least one of the two or more membranes filters out particles having a molecular weight greater than 10 kDa, and wherein the ultrafiltering is performed prior to the addition of the hydrolysate to a cell culture medium.

10. The method according to claim 9, comprising the step of ultrafiltering the hydrolysate through at least one membrane that filters out particles having a molecular weight greater than 5 kDa.

11. The method according to claim 10, comprising the step of ultrafiltering the hydrolysate through two membranes, wherein the first membrane filters out particles having a molecular weight greater than 10 kDa, and wherein the second membrane filters out particles having a molecular weight greater than 5 kDa.

12. The method according to claim 1, further comprising the step of ultrafiltering the hydrolysate through one or more membranes, wherein at least one of the one or more membranes filters out particles having a molecular weight greater than 10 kDa; wherein the heating and ultrafiltering are performed prior to the addition of the hydrolysate to a cell culture medium.

13. The method according to claim 12, wherein at least one of the one or more membranes filters out particles having a molecular weight greater than 5 kDa.

14. The method according to claim 12, comprising the step of ultrafiltering the hydrolysate through two or more membranes, wherein at least one membrane filters out particles having a molecular weight greater than 10 kDa, and wherein at least one membrane filters out particles having a molecular weight greater than 5 kDa.
